(19) United States
(12) Patent Application Publication
(10) Pub. No.: US 2016/0073905 A1
(43) Pub. Date: Mar. 17, 2016

(54) BLOOD PRESSURE MEASURING DEVICE AND BLOOD PRESSURE MEASURING METHOD

(71) Applicant: SEIKO EPSON CORPORATION, Tokyo (JP)

(72) Inventors: Kiyoaki MURAI, Matsumoto-shi (JP); Hiromitsu MIZUKAMI, Matsumoto-shi (JP)

(21) Appl. No.: 14/836,353

(22) Filed: Aug. 26, 2015

(30) Foreign Application Priority Data
Sep. 12, 2014 (JP) 2014-186041

(51) Int. Cl. A61B 5/0205; A61B 5/00; A61B 5/107; A61B 5/021

(52) U.S. Cl. CPC A61B 5/0205; A61B 5/021; A61B 5/7278; A61B 5/1075; A61B 5/7425

(57) ABSTRACT

Measurement data of a blood vessel is generated based on a measurement result of the blood vessel obtained by a blood vessel diameter measurement unit, and a breathing period estimating section estimates a breathing period corresponding to a breathing cycle based on the measurement data. A blood pressure calculating section calculates blood pressure relevant to the blood vessel based on the measurement data. An average calculating section calculates the average of the blood pressure calculated by the blood pressure calculating section in the breathing period estimated by the breathing period estimating section.

### BACKGROUND

1. Technical Field

The present invention relates to a blood pressure measuring device and the like.

2. Related Art

One of the important parameters for checking the condition of the human body is blood pressure, and blood pressure measurement in the medical field is one of the indispensable elements. For the blood pressure measuring method, various techniques are disclosed in the related art. For example, there is a technique of placing an ultrasonic probe on the skin surface on the blood vessel, calculating a blood vessel diameter from the reflected wave of the ultrasonic wave, and measuring blood pressure based on the relationship between the blood pressure and the blood vessel diameter (for example, refer to JP-A-2005-28123). In addition, a technique of measuring and displaying blood pressure for each beat of the cardiac cycle (for example, refer to JP-A-2008-12230), a technique of recognizing the last stage of breathing using a respiration sensor and adopting and displaying the measurement value of a pressure sensor at the timing of the last stage of breathing as a measurement result (for example, refer to JP-A-2010-200901), and the like are also known.

Incidentally, it is known that blood pressure varies with breathing (hereinafter, referred to as “respiratory variation”). In the technique disclosed in JP-A-2005-28123 or JP-A-2008-12230, however, components of the respiratory variation are included in the measured blood pressure value. Therefore, for example, when the blood pressure value is digitally displayed at each measurement timing (for example, every second), the displayed value is not stable due to the influence of respiratory variation. For this reason, it has been difficult to read an appropriate blood pressure value in consideration of the influence of respiratory variation. Even if the reading of the average value of measurement values of a predetermined number of times (for example, 10 times) is determined in order to reduce the influence of respiratory variation, the time required for the measurement of the predetermined number of times does not necessarily match the period of the respiratory variation.

On the other hand, in the technique disclosed in JP-A-2010-200901, it is possible to reduce the influence of respiratory variation by setting the last stage of breathing as the measurement timing. However, since an additional respiration sensor should be used, there is a problem in terms of the manufacturing cost. In recent years, there are many cases requiring a long time for blood pressure measurement while living daily life at home. In this case, miniaturization of a blood pressure measuring device is strongly demanded. In the configuration that requires a respiration sensor, however, it is also difficult to meet the demand of such miniaturization.

### SUMMARY

An advantage of some aspects of the invention is to realize blood pressure measurement with the reduced influence of respiratory variation without an increase in the number of hardware components, such as a respiration sensor.

A first aspect of the invention is directed to a blood pressure measuring device including: a measurement data generation unit that generates measurement data of a blood vessel based on a measurement result of the blood vessel obtained by a measurement unit; a breathing period determination unit that determines a breathing period corresponding to a breathing cycle based on the measurement data; a blood pressure calculation unit that calculates blood pressure of the blood vessel based on the measurement data; and an average calculation unit that calculates an average of the blood pressure of the blood vessel in the breathing period.

The breathing cycle means a set of exhalation and inhalation, and the breathing period is a period corresponding to the breathing cycle.

According to the first aspect of the invention, it is possible to calculate the blood pressure based on the measurement data of the blood vessel and to estimate a breathing period corresponding to the breathing cycle. In addition, it is possible to calculate the average of the blood pressure in the estimated breathing period. Therefore, it is possible to realize blood pressure measurement with the reduced influence of respiratory variation without an increase in the number of hardware components, such as a respiration sensor.

A second aspect of the invention is directed to the blood pressure measuring device according to the first aspect of the invention, in which the average calculation unit calculates an average of the blood pressure of the blood vessel in a plurality of breathing periods.

According to the second aspect of the invention, for example, it is possible to calculate the average value of the blood pressure in consecutive breathing periods.

A third aspect of the invention is directed to the blood pressure measuring device according to the first or second aspect of the invention, in which the average calculation unit calculates an average of diastolic blood pressure or systolic blood pressure of the blood vessel.

According to the third aspect of the invention, it is possible to calculate the average value of the diastolic blood pressure or the systolic blood pressure.

A fourth aspect of the invention is directed to the blood pressure measuring device according to any one of the first to third aspects of the invention, which further includes a display control unit that continuously displays the blood pressure calculated by the average calculation unit by inserting a predetermined switching display whenever the breathing period for calculation of the blood pressure is switched.

When performing monitor display of the blood pressure value, the display of the blood pressure value is updated every breathing period. However, a situation is also considered in which the displayed value appears not to be changed at all. In such a scene, it is believed that the operator cannot immediately determine whether or not the measurement is being successfully performed as long as the operator sees the monitor display.

However, according to the fourth aspect of the invention, a switching display is inserted at the switching timing of the breathing period. Therefore, even if the same value is displayed in consecutive breathing periods, it is clear that the display has been updated. As a result, the aforementioned concerns are eliminated.

A fifth aspect of the invention is directed to a blood pressure measuring method including: generating measurement data of a blood vessel based on a measurement result of the blood vessel obtained by a measurement unit; determining a breathing period corresponding to a breathing cycle based on the measurement data; calculating blood pressure of the blood vessel based on the measurement data; and calculating an average of the blood pressure of the blood vessel in the breathing period.

According to the fifth aspect of the invention, the same effect as in the first invention is obtained.

A sixth aspect of the invention is directed to the blood pressure measuring method according to the fifth aspect of the invention, in which the calculation of the average includes calculating an average of the blood pressure of the blood vessel in a plurality of breathing periods.

According to the sixth aspect of the invention, the same effect as in the second invention is obtained.

### BRIEF DESCRIPTION OF THE DRAWINGS

The invention will be described with reference to the accompanying drawings, wherein like numbers reference like elements.

FIG. 1 is a diagram showing an example of the system configuration of a blood pressure measuring device.

FIG. 2 is a diagram for explaining a method of calculating the relative position of the blood vessel to be measured and the blood vessel diameter from the reflected wave of the ultrasonic wave.

FIG. 3 is a diagram for explaining the principle of reducing the influence of respiratory variation in blood pressure measurement.

FIG. 4 is a diagram showing a display example of a measurement result.

FIG. 5 is a block diagram showing an example of the functional configuration of an ultrasonic blood pressure measuring device.

FIG. 6 is a flowchart for explaining the flow of the process related to the measurement of biological information by a measurement control device.

FIG. 7 is a flowchart continued from FIG. 6.

### DESCRIPTION OF EXEMPLARY EMBODIMENTS

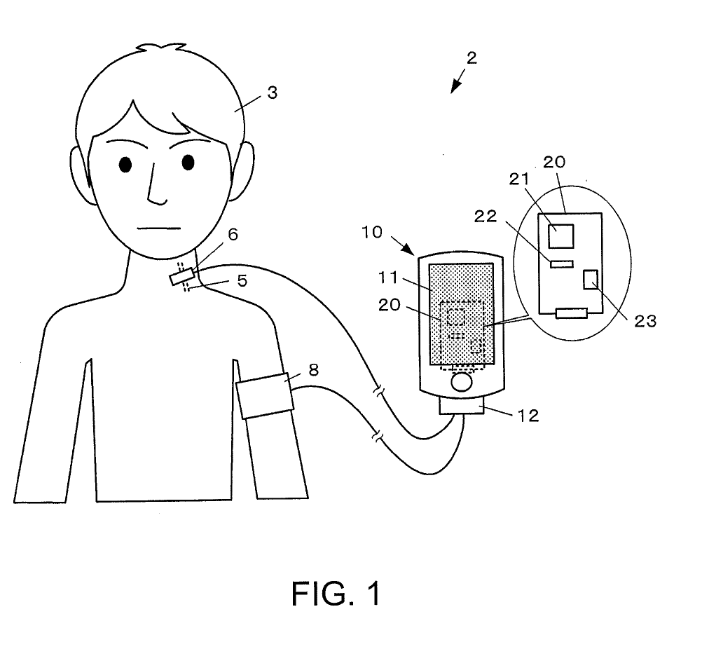

FIG. 1 is a diagram showing an example of the configuration of a blood pressure measuring device in the present embodiment.

A blood pressure measuring device 2 is a system for measuring biological information by emitting an ultrasonic wave to a blood vessel to be measured and analyzing the reflected wave from the blood vessel. In the present embodiment, it is assumed that a blood vessel diameter and blood pressure are measured as biological information. However, it is also possible to measure other kinds of biological information.

The blood pressure measuring device 2 of the present embodiment includes an ultrasonic probe 6 that is always attached to a part to be measured (in the present embodiment, a blood vessel to be measured: carotid artery 5) of a person to be measured 3, a calibration sphygmomanometer 8, and a measurement control device 10.

The ultrasonic probe 6 performs transmission and reception of an ultrasonic wave, generates a reception signal corresponding to the strength of the received reflected wave, and outputs the generated reception signal to the measurement control device 10. Such a function can be realized by the technique according to the known ultrasonic measurement.

In the present embodiment, immediately above the left carotid artery 5, at least one of a plurality of ultrasonic transducer columns arrayed in the ultrasonic probe 6 is attached so as to straddle the short axis of the blood vessel while crossing the long axis of the blood vessel. “Immediately above” referred to herein is used, for easy understanding, in the operating manual expression when operating the ultrasonic probe 6. To be precise, “immediately above” refers to the positional relationship in which the carotid artery is located on the straight line of irradiation of ultrasonic waves that are emitted from the ultrasonic transducers arrayed in the ultrasonic probe 6.

The calibration sphygmomanometer 8 measures blood pressure for calculating the relationship between blood pressure and a blood vessel diameter (hereinafter, referred to as “blood pressure-blood vessel diameter relationship”), and outputs the measurement result to the measurement control device 10. In the present embodiment, a cuff type electronic sphygmomanometer is used. However, other types of sphygmomanometers, such as a tonometer capable of measuring blood pressure every beat, may be used. In addition, the calibration sphygmomanometer 8 can be appropriately removed after calculating the blood pressure-blood vessel diameter relationship by performing calibration before the start of measurement.

The measurement control device 10 is a computer for realizing a function of measuring the biological information of the circulatory system including blood vessels. In the present embodiment, the measurement control device 10 realizes 1) a blood vessel position determining function for determining the relative position of the blood vessel with respect to the ultrasonic probe 6 based on the reception signal obtained from the ultrasonic probe 6, 2) a measurement execution function for executing the measurement of biological information using the ultrasonic probe 6 based on the determination result of the blood vessel position, 3) a data logging function for periodically executing measurement until the predetermined measurement end conditions are satisfied and storing the measurement result, and 4) a monitor display function for displaying measurement results in a sequential manner.

Specifically, the measurement control device 10 includes a touch panel 11, an interface circuit 12, a control board 20, and an internal battery (not shown). A central processing unit (CPU) 21 executes a program stored in an IC memory 22 mounted on the control board 20, and the measurement control device 10 realizes the above-described blood vessel position determining function, measurement execution function, data logging function, and monitor display function based on the input data from the ultrasonic probe 6 or the calibration sphygmomanometer 8 connected through the interface circuit 12.

In the example shown in FIG. 1, communication connection between the ultrasonic probe 6 or the calibration sphygmomanometer 8 and the measurement control device 10 is realized by a cable. However, the communication connection between the ultrasonic probe 6 or the calibration sphygmomanometer 8 and the measurement control device 10 may also be realized by wireless communication by mounting a short-distance radio device 23 or the like on the control board 20. The measurement control device 10 of the present embodiment can be configured as a portable information terminal, such as a smartphone or a wearable computer capable of executing an application program. Alternatively, the measurement control device 10 of the present embodiment may be configured as a stationary device or as an external device connected through a mobile phone network, the Internet, or a local area network (LAN).

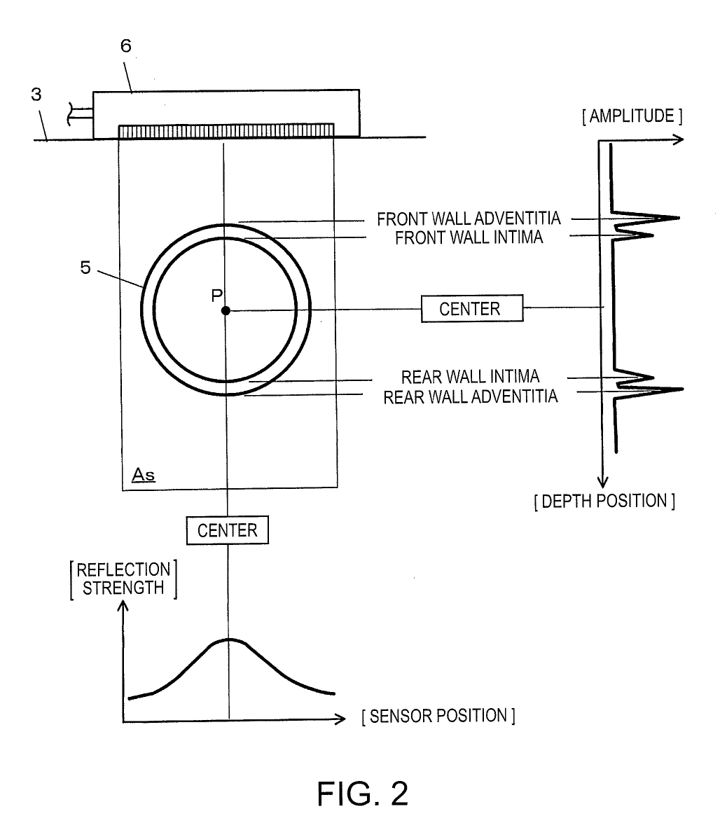

FIG. 2 is a diagram for explaining a method of determining the relative position of the blood vessel to be measured from the reflected wave of the ultrasonic wave.

The blood vessel position is expressed by the coordinate values of two axes of a “depth position” and a “sensor position”. The “depth position” is a position when the traveling direction of the ultrasonic beam from the ultrasonic probe 6 to the inside of the body is set as an axis, and the “sensor position” is a position when the arrangement direction of ultrasonic transducers (ultrasonic sensors) is set as an axis. In the present embodiment, it is assumed that the blood vessel position is represented by the position of the center of the short-axis section of the blood vessel (blood vessel center P). Needless to say, the front wall (blood vessel wall on a side close to the ultrasonic probe 6) or the rear wall of the blood vessel can also be used. However, since the blood vessel wall continuously expands and contracts due to beating, it is desirable to set the center position as a reference so that the blood vessel positions can be accurately compared. Even if the blood vessel center position is used, the position may change slightly according to the timing of beating. Therefore, when determining the blood vessel position, a position in the diastole (referred to in blood pressure) in which the blood vessel diameter is the minimum or a position in the systole in which the blood vessel diameter is the maximum is adopted.

In the example shown in FIG. 2, since a case is assumed in which the ultrasonic wave is transmitted in a linear direction from the respective ultrasonic transducers of the ultrasonic probe 6, a range that the ultrasonic wave reaches, that is, an observation region As (the same as a scanning region by the ultrasonic wave) is a rectangular shape. However, in a case in which the ultrasonic wave can be transmitted in an oblique direction, the observation region As becomes an approximately trapezoidal shape or fan shape due to the extension of the lower side in FIG. 2.

In addition, as a method of detecting the blood vessel center P, the distribution of the reflection strength of each ultrasonic transducer and the amplitude strength information in the depth direction are used. Specifically, the reflection strength increases as the amount of transmission waves of ultrasonic waves that are emitted perpendicular to the blood vessel section increases. Therefore, the position of the ultrasonic transducer indicating the maximum of the distribution of the reflection strength of each ultrasonic transducer is assumed to be the sensor position coordinate value of the blood vessel center P. Then, vascular front and rear walls are detected from the peak position of the signal strength of amplitude data in the depth direction at the sensor position, and the intermediate position is assumed to be the depth position coordinate value of the blood vessel center P. Needless to say, the method of detecting the blood vessel center P is not limited thereto, and other known methods can be appropriately adopted.

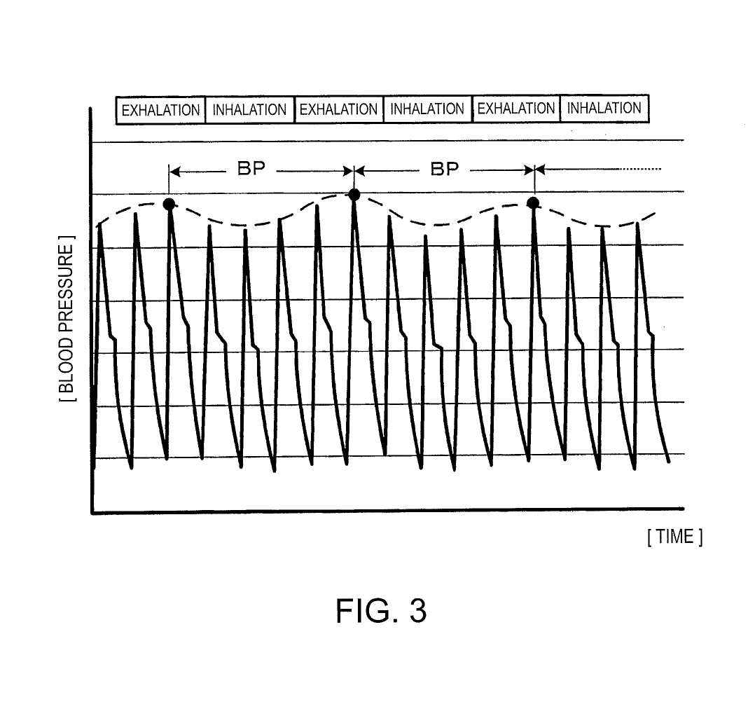

FIG. 3 is a diagram for explaining the principle of reducing the influence of respiratory variation in the blood pressure measurement in the present embodiment.

As shown by the blood pressure waveform, blood pressure increases or decreases under the influence of breathing. Specifically, blood pressure decreases at the time of inhalation and increases at the time of exhalation at periods of about 3 to 6 seconds. In the case of a healthy person, it is said that the variation width is less than 5 mmHg in normal breathing and 20 mmHg in deep breathing.

In the present embodiment, in order to reduce the influence of such respiratory variation of blood pressure, a breathing period BP (combination of one inhalation and one exhalation) corresponding to the breathing cycle is estimated from changes in blood pressure, blood pressure values in a predetermined number of consecutive breathing periods are averaged, and the average value is adopted as a measurement value. Although the following explanation is given with a predetermined number of breathing periods as one period, the predetermined number of breathing periods may be two or more periods.

Specifically, focusing on the systolic blood pressure, the systolic blood pressure increases over the exhalation from the inhalation, and a rising peak occurs in the middle of the exhalation. Then, the systolic blood pressure value decreases over the inhalation from the last stage of the exhalation, and a falling peak occurs in the middle of the inhalation. Therefore, the rising peak or falling peak of the systolic blood pressure value is detected, a period until the next corresponding peak (the next rising peak in the case of rising peak, and the next falling peak in the case of falling peak) is detected is determined to be one breathing cycle, and this period is set as a breathing period. Then, the average of the systolic blood pressure between the corresponding peaks is calculated, and the calculated average is set as a measurement value of the systolic blood pressure in the breathing period. Similarly, the average of the diastolic blood pressure between the corresponding peaks is calculated, and the calculated average is set as a measurement value of the diastolic blood pressure in the breathing period.

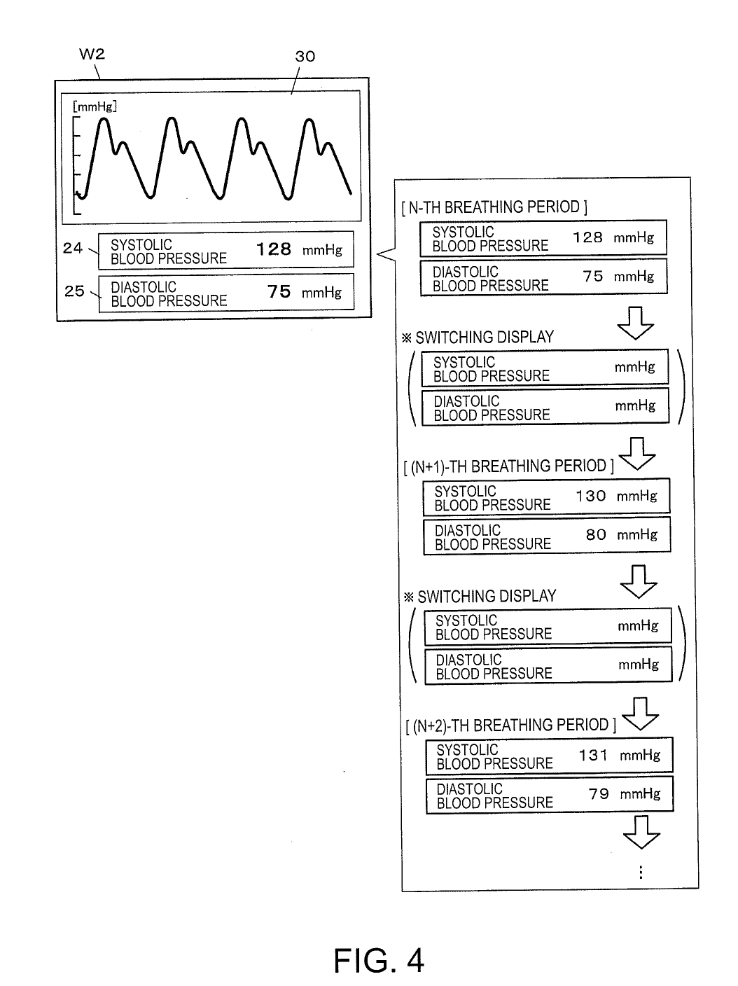

FIG. 4 is a diagram showing a display example of the measurement result in the present embodiment.

When the measurement is started, the measurement control device 10 displays the monitor screen W2 on the touch panel 11. A blood pressure waveform display portion 30, a first measurement value display portion 24, and a second measurement value display portion 25 are included in the monitor screen W2.

Blood pressure waveforms that have been continuously measured and calculated are displayed in the blood pressure waveform display portion 30.

The measurement value of systolic blood pressure in the latest breathing period (average value of the systolic blood pressure in the breathing period) is displayed in the first measurement value display portion 24, and the measurement value of diastolic blood pressure in the latest breathing period (average value of the diastolic blood pressure in the breathing period) is displayed in the second measurement value display portion 25.

More specifically, blood pressure value display during one breathing period is the same, but a switching display is inserted when updating the blood pressure value display due to breathing period switching. In the present embodiment, as the switching display, a “blank display” in which no measurement value is displayed is inserted. For example, the measurement value from the start of measurement to the n-th (n is a natural number) breathing period is displayed for a predetermined period of time, and then no measurement value is displayed for about 0.5 seconds, for example, and the measurement value in a subsequent (n+1)-th breathing period is displayed for a predetermined period of time. Needless to say, the “blank display” is also performed similarly before the measurement value of the next (n+2)-th breathing period is displayed.

When the influence of respiratory variation is reduced, there may be a case in which the value displayed in the first measurement value display portion 24 and the value displayed in the second measurement value display portion 25 are not changed at all even after breathing period switching. However, if a switching display is not inserted, the operator cannot identify whether the device has failed or there is no variation in the measurement value by chance. According to the present embodiment, since it becomes clear that the display of the measurement value has been updated through the presence of the switching display, such concerns are eliminated.

In addition to the blank display, the form of the switching display can be appropriately set. For example, it is possible to temporarily reverse the black and white display, or it is possible to appropriately adopt a known transition technique in video editing techniques.

### Explanation of Functional Configuration

Next, the functional configuration for realizing the present embodiment will be described.

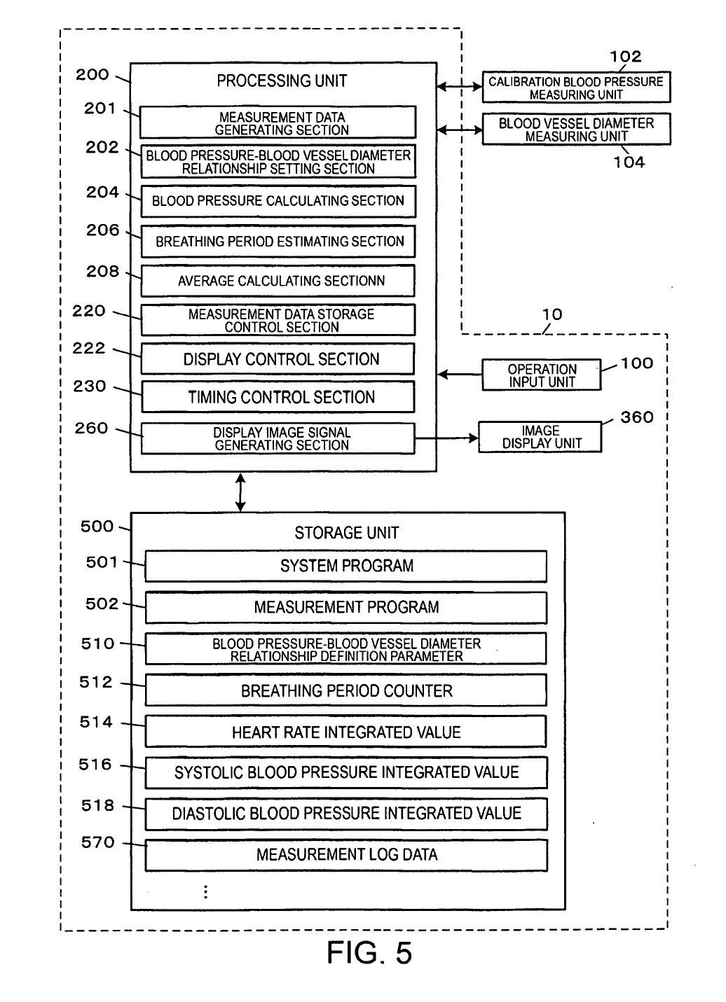

FIG. 5 is a block diagram showing an example of functional configuration of the blood pressure measuring device 2 of the present embodiment. The blood pressure measuring device 2 includes an operation input unit 100, a processing unit 200, an image display unit 360, and a storage unit 500 that are included in the measurement control device 10. In addition, the blood pressure measuring device 2 includes a calibration blood pressure measuring unit 102 and a blood vessel diameter measuring unit 104 that are connected to the measurement control device 10.

The operation input unit 100 receives various kinds of operation input performed by the operator, and outputs an operation input signal corresponding to the operation input to the processing unit 200. The operation input unit 100 can be realized by a button switch, a lever switch, a dial switch, a track pad, a mouse, and the like. The touch panel 11 shown in FIG. 1 corresponds to the operation input unit 100.

The calibration blood pressure measuring unit 102 measures calibration blood pressure for calculating the blood pressure-blood vessel diameter relationship, and outputs the measurement result to the processing unit 200. The calibration sphygmomanometer 8 shown in FIG. 1 corresponds to the calibration blood pressure measuring unit 102.

The blood vessel diameter measuring unit 104 performs measurement for calculating the blood vessel diameter using an ultrasonic wave. The ultrasonic probe 6 shown in FIG. 1 corresponds to the blood vessel diameter measuring unit 104, and outputs the reflection strength of each ultrasonic transducer, amplitude data in the depth direction, and the like to the processing unit 200. The blood vessel diameter measuring unit 104 may have a function of calculating the blood vessel diameter.

The processing unit 200 is realized, for example, by a microprocessor, such as a CPU or a graphics processing unit (GPU), or an electronic component, such as an application specific integrated circuit (ASIC) or an IC memory. The processing unit 200 controls the input and output of data between the respective functional units, and performs overall control of the blood pressure measuring device 2 and the measurement control device 10 by executing various kinds of arithmetic processing based on a predetermined program or various kinds of data. The control board 20 shown in FIG. 1 corresponds to the processing unit 200.

In the present embodiment, the processing unit 200 includes a measurement data generating section 201, a blood pressure-blood vessel diameter relationship setting section 202, a blood pressure calculating section 204, a breathing period estimating section 206, and an average calculating section 208. In addition, the processing unit 200 includes a measurement data storage control section 220, a display control section 222, a timing control section 230, and a display image signal generating section 260.

The measurement data generating section 201 generates measurement data of a blood vessel. In the present embodiment, the blood vessel center P of the carotid artery 5 to be measured is detected based on the data (received data of the reflected wave of each ultrasonic transducer) obtained from the blood vessel diameter measuring unit 104, and the blood vessel diameter is calculated by detecting the blood vessel wall from the received data of the reflected wave passing through the blood vessel center P (refer to FIG. 2).

The blood pressure-blood vessel diameter relationship setting section 202 sets the relationship for calculating the biological information of the measurement target in the present embodiment. In other words, the blood pressure-blood vessel diameter relationship setting section 202 performs processing relevant to the calibration. Specifically, systolic blood pressure, diastolic blood pressure, systolic blood vessel diameter, and diastolic blood vessel diameter are calculated based on the blood pressure obtained by the calibration blood pressure measuring unit 102 and the blood vessel diameter continuously obtained by the blood vessel diameter measuring unit 104, and the blood pressure-blood vessel diameter relationship including a stiffness parameter β is calculated and set. Such a function can be realized by appropriately using a known technique.

The blood pressure calculating section 204 calculates blood pressure at predetermined periods (for example, every second) by substituting the data obtained by the blood vessel diameter measuring unit 104 into the blood pressure-blood vessel diameter relationship, and calculates the systolic blood pressure and the diastolic blood pressure for each heart beat.

The breathing period estimating section 206 estimates a breathing period based on the data obtained by the blood vessel diameter measuring unit 104. Specifically, the peak of the systolic blood pressure value (or the peak of diastolic blood pressure value) is detected from the variation of the blood pressure value calculated based on the data obtained by the blood vessel diameter measuring unit 104, and a period between two consecutive corresponding peaks is regarded as one breathing cycle and the period determined by the peaks is assumed to be one breathing period.

The average calculating section 208 calculates the average of the blood pressure in a plurality of consecutive breathing periods. In the present embodiment, in order to simplify the explanation, the average of the systolic blood pressure and the average of the diastolic blood pressure in one breathing period are calculated, and these averages are calculated as measurement values in the breathing period.

Specifically, when the peak of the systolic blood pressure value is detected by the breathing period estimating section 206, a heart rate and systolic blood pressure and diastolic blood pressure for each heart beat are integrated, and the integrated value of the systolic blood pressure and the integrated value of the diastolic blood pressure are averaged by the heart rate between the peaks when the next peak of the systolic blood pressure value is detected. In the present embodiment, the average is calculated every single breathing period. However, the average may be calculated every plural consecutive breathing periods, such as every two consecutive breathing periods or every three consecutive breathing periods.

The measurement data storage control section 220 registers and stores the blood pressure value calculated by the blood pressure calculating section 204 or the value (the average value of systolic blood pressure and the average value of diastolic blood pressure) calculated by the average calculating section 208 in the measurement log data 570 of the storage unit 500 so as to match the date and time information.

The display control section 222 performs display control of the monitor screen W2 (refer to FIG. 4). In the present embodiment, whenever a new average value is calculated by the average calculating section 208 every breathing period switching, control for the insertion of a switching display is performed. Specifically, the display control is performed such that switching to a blank display is performed by removing the display of the average value that has been displayed until then, the blank display is performed for a predetermined period of time, and then the newly calculated average value is displayed.

The timing control section 230 performs control relevant to the timing. In the present embodiment, measurement or management of the current date and time, counting of the measurement period, and the like are performed. It is needless to say that other timer processing and the like can also be appropriately performed.

The display image signal generating section 260 is realized, for example, by a processor such as a GPU or a digital signal processor (DSP), a program such as a video signal IC or video CODEC, or an IC memory for drawing frames such as a frame buffer. The display image signal generating section 260 generates an image signal for displaying a monitor screen W2 (refer to FIG. 4) and the like, and outputs the image signal to the image display unit 360.

The image display unit 360 displays various images based on the image signal input from the display image signal generating section 260. For example, the image display unit 360 can be realized by an image display device, such as a flat panel display, a cathode ray tube (CRT), a projector, or a head-mounted display. In the present embodiment, the touch panel 11 shown in FIG. 1 corresponds to the image display unit 360.

The storage unit 500 is realized by a storage medium, such as an IC memory, a hard disk, or an optical disc, and stores various programs or various kinds of data, such as data during the calculation process of the processing unit 200. In FIG. 1, the IC memory 22 mounted on the control board 20 corresponds to the storage unit 500. In addition, the connection between the processing unit 200 and the storage unit 500 is not limited to a connection using an internal bus circuit in the device, and may be realized by using a communication line, such as a local area network (LAN) or the Internet. In this case, the storage unit 500 maybe realized by a separate external storage device from the measurement control device 10.

The storage unit 500 stores a system program 501, a measurement program 502, a blood pressure-blood vessel diameter relationship definition parameter 510, and a breathing period counter 512. In addition, the storage unit 500 stores a heart rate integrated value 514, a systolic blood pressure integrated value 516, a diastolic blood pressure integrated value 518, and measurement log data 570. Needless to say, programs or data other than these, for example, a counter for counting the time or a flag can also be appropriately stored.

The processing unit 200 executes the system program 501, thereby realizing a basic input/output function as a computer.

The processing unit 200 executes the measurement program 502, thereby realizing the measurement data generating section 201, the blood pressure-blood vessel diameter relationship setting section 202, the blood pressure calculating section 204, the breathing period estimating section 206, the average calculating section 208, the measurement data storage control section 220, the display control section 222, the timing control section 230, and the display image signal generating section 260.

When realizing these functional units with hardware, such as electronic circuits, apart of the program for realizing the function can be omitted.

The blood pressure-blood vessel diameter relationship definition parameter 510 includes various parameter values that define the blood pressure-blood vessel diameter relationship using the stiffness parameter β. For example, systolic blood pressure, diastolic blood pressure, systolic blood vessel diameter, diastolic blood vessel diameter, the stiffness parameter β, and the like are included.

The breathing period counter 512 is managed by the breathing period estimating section 206, and indicates the number of breathing periods that are used as periods for calculating the average value of the blood pressure. The counter is reset to “0” when the counter value reaches a predetermined value (the number of breathing periods that are essential when the average calculating section 208 newly calculates the average value), and is incremented by “1” whenever the breathing period is estimated and determined. In addition, the average calculating section 208 of the present embodiment calculates an average value when the counter reaches a specified value (in the present embodiment, “1”).

The heart rate integrated value 514 is an integrated value of the heart rate in a period of calculating the average value of the blood pressure. The heart rate integrated value 514 is reset to “0” when the average value is calculated by the average calculating section 208.

The systolic blood pressure integrated value 516 and the diastolic blood pressure integrated value 518 are an integrated value of the systolic blood pressure and an integrated value of the diastolic blood pressure, respectively, for each heart beat in a period until the new average value is calculated. The systolic blood pressure integrated value 516 and the diastolic blood pressure integrated value 518 are reset to “0” when the average blood pressure is calculated by the average calculating section 208.

In the measurement log data 570, measurement results are stored in time series. In the present embodiment, the blood pressure value calculated by the blood pressure calculating section 204 is stored in time series so as to match the recording date and time. The average value of the systolic blood pressure and the average value of the diastolic blood pressure that have been calculated by the average calculating section 208 are also stored in time series so as to match the recording date and time.

### Explanation of Operations

Next, the operation of the blood pressure measuring device 2 will be described.

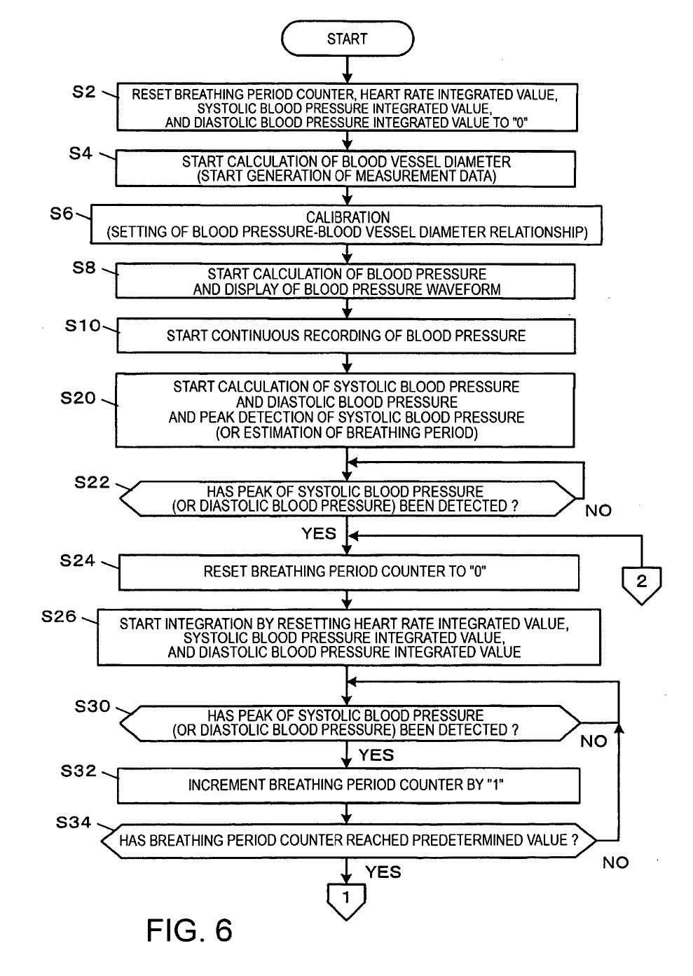

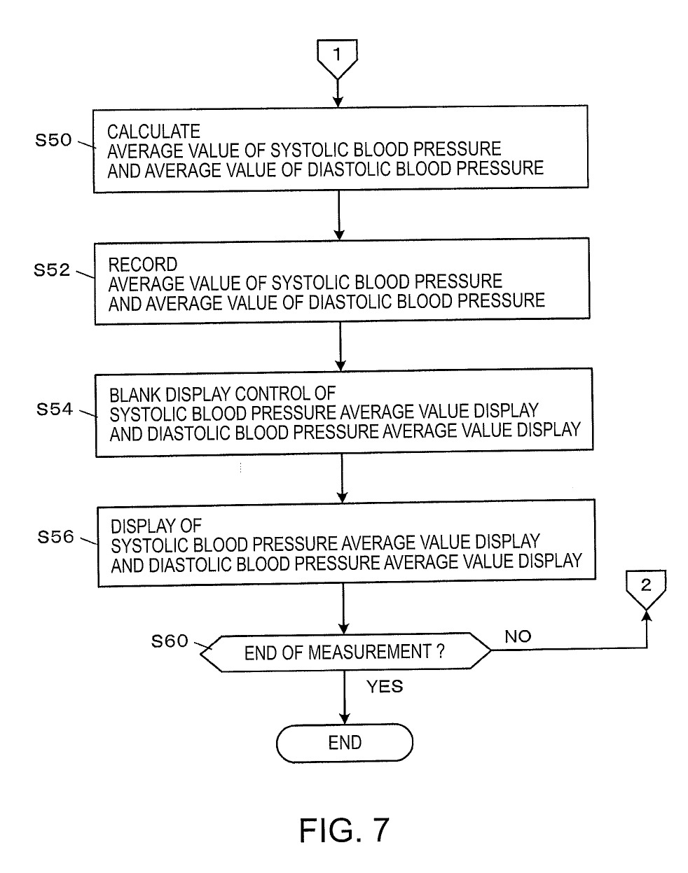

FIGS. 6 and 7 are flowcharts for explaining the flow of the process related to the measurement of biological information by the measurement control device 10. As shown in FIG. 6, first, the measurement control device 10 initializes the breathing period counter 512, the heart rate integrated value 514, the systolic blood pressure integrated value 516, and the diastolic blood pressure integrated value 518 by resetting these to “0” (step S2). Then, the measurement control device 10 starts the acquisition of a reception signal of the reflected wave of the ultrasonic wave from the ultrasonic probe 6, thereby starting the continuous calculation of the blood vessel diameter of the carotid artery 5 (step S4). That is, the generation of measurement data of the blood vessel is started based on the measurement result using the ultrasonic wave.

Then, the measurement control device 10 sets a blood pressure-blood vessel diameter relationship by performing calibration processing (step S6). Since the setting of the blood pressure-blood vessel diameter relationship can be realized in the same manner as in the related art, explanation herein will be omitted.

Then, the calculation of blood pressure using the blood pressure-blood vessel diameter relationship and the waveform display of the blood pressure calculated in the monitor screen W2 are started (step S8), and control to sequentially record the calculated blood pressure value in the measurement log data 570 is started (step S10).

Then, the measurement control device 10 starts the calculation of systolic blood pressure and diastolic blood pressure for each heart beat and the peak detection of the systolic blood pressure (or the diastolic blood pressure) (step S20). It is possible to estimate the breathing period by the peak detection.

Then, when the peak of the systolic blood pressure (or the diastolic blood pressure) is detected (YES in step S22), the measurement control device 10 resets the breathing period counter 512 to “0” (step S24).

Then, the heart rate integrated value 514, the systolic blood pressure integrated value 516, and the diastolic blood pressure integrated value 518 are reset to “0” to start the integration of each of the values (step S26). Hereinafter, whenever the systolic blood pressure is detected, the measurement control device 10 adds “1” to the heart rate integrated value 514, and adds the value of the latest systolic blood pressure to the systolic blood pressure integrated value 516. In addition, when the diastolic blood pressure is detected, the measurement control device 10 adds the value of the latest diastolic blood pressure to the diastolic blood pressure integrated value 518.

Then, when the peak of the systolic blood pressure (or the diastolic blood pressure) is detected again (YES in step S30), the measurement control device 10 determines a period between the peak detected in step S22 and the peak detected this time to be one breathing cycle, regards the period of one breathing cycle as a breathing period, and increments the breathing period counter 512 by “1” (step S32).

Then, when the breathing period counter 512 reaches a predetermined value (in the present embodiment, “1”) (YES in step S34), the measurement control device 10 calculates the average value of the systolic blood pressure by dividing the systolic blood pressure integrated value 516 by the heart rate integrated value 514, and similarly calculates the average value of the diastolic blood pressure by dividing the diastolic blood pressure integrated value 518 by the heart rate integrated value 514, as shown in FIG. 7 (step S50). Since these are the latest measurement values, the measurement control device 10 records the average value of the systolic blood pressure and the average value of the diastolic blood pressure in the measurement log data 570 (step S52).

Then, the measurement control device 10 performs “blank display” in the first measurement value display portion 24 and the second measurement value display portion 25 (step S54), so that the average value of the latest systolic blood pressure is displayed in the first measurement value display portion 24 and the average value of the latest diastolic blood pressure is displayed in the second measurement value display portion 25 (step S56).

Then, the measurement control device 10 determines whether or not the measurement end conditions are satisfied (step S60). The measurement end conditions can be appropriately set. For example, the measurement end conditions include an elapsed time from the start of measurement, the number of times of measurement, and the detection of a predetermined measurement end operation.

When the measurement end conditions are not satisfied (NO in step S60), the process returns to step S24. Since the peak detected in step S30 is also the start timing of the next breathing period, preparation for calculating the next average value is started (steps S24 to S26: FIG. 6).

When the measurement end conditions are satisfied (YES in step S60), the measurement control device 10 ends the series of processing.

As described above, according to the present embodiment, by estimating a breathing period without an increase in the number of hardware components, such as a respiration sensor, by detecting the peak of blood pressure, calculating the average value of the blood pressure during the breathing period, and determining this as a measurement value, it is possible to realize blood pressure measurement with the reduced influence of respiratory variation.

Embodiments of the invention are not limited to the above, and constituent components can be appropriately added, omitted, and changed.

For example, when calculating the average of the systolic blood pressure and the average of the diastolic blood pressure in a plurality of consecutive breathing periods, the plurality of consecutive breathing periods may be set by shifting breathing periods for the calculation one by one. Specifically, assuming that the number of breathing periods for calculating the average of the systolic blood pressure and the average of the diastolic blood pressure is N (N≧2), each average is calculated in the first to N-th breathing periods and then the average is calculated in the second to (N+1)-th breathing periods. Then, the average is calculated in the third to (N+2)-th breathing periods. Thus, N breathing periods are set by being shifted one by one. In this manner, new calculation and display are performed whenever breathing period switching occurs.

The entire disclosure of Japanese Patent Application No. 2014-186041 filed on 9/12/2014 is expressly incorporated by reference herein.

What is claimed is:

1. A blood pressure measuring device, comprising:
a breathing period determination unit that determines a breathing period corresponding to a breathing cycle based on measurement data of a blood vessel obtained by a measurement unit;
a first calculation unit that calculates blood pressure of the blood vessel based on the measurement data; and
a second calculation unit that calculates blood pressure based on a plurality of values of the blood pressure calculated by the first calculation unit in the breathing period.

2. The blood pressure measuring device according to claim 1,
wherein the second calculation unit calculates an average of the plurality of values of the blood pressure calculated by the first calculation unit.

3. The blood pressure measuring device according to claim 1,
wherein the second calculation unit calculates an average of the plurality of values of the blood pressure calculated by the first calculation unit in a plurality of breathing periods.

4. The blood pressure measuring device according to claim 1,
wherein the second calculation unit calculates an average of diastolic blood pressure or systolic blood pressure of the blood vessel.

5. The blood pressure measuring device according to claim 1,
wherein the measurement data is a blood vessel diameter of the blood vessel, and
the breathing period determination unit determines the breathing cycle from a periodic variation in the blood vessel diameter.

6. The blood pressure measuring device according to claim 1, further comprising:
a display control unit that continuously displays the blood pressure calculated by the second calculation unit by inserting a predetermined switching display whenever the breathing period for calculation of the blood pressure is switched.

7. A blood pressure measuring method, comprising:
determining a breathing period corresponding to a breathing cycle based on measurement data of a blood vessel obtained by a measurement unit;
calculating blood pressure of the blood vessel based on the measurement data; and
calculating blood pressure based on a plurality of values of the blood pressure calculated in the breathing period.

8. The blood pressure measuring method according to claim 7,
wherein the calculation of the blood pressure includes calculating an average of a plurality of values of the blood pressures calculated in the breathing period.

9. The blood pressure measuring method according to claim 7,
wherein the calculation of the blood pressure includes calculating an average of a plurality of values of the blood pressures calculated in a plurality of breathing periods.
